(19) United States
(12) Patent Application Publication
(10) Pub. No.: US 2011/0207724 A1
(43) Pub. Date: Aug. 25, 2011

(54) PHARMACEUTICAL FORMULATIONS AND METHODS OF USE WHICH COMBINE NON-STEROIDAL ANTI-INFLAMMATORY COMPOUNDS WITH ANIT-HYPERTENSIVE COMPOUNDS

(76) Inventors: Peter C. Hoyle, Lovettsville, VA (US); Paul Waymack, Washington, DC (US)

(21) Appl. No.: 12/990,724

(22) PCT Filed: May 22, 2009
(86) PCT No.: PCT/US09/44966

§ 371 Date: Dec. 2, 2010

Related U.S. Application Data
(60) Provisional application No. 61/056,789, filed on May 28, 2008.
(60) Provisional application No. 61/097,972, filed on Sep. 18, 2008.

(51) Int. Cl. A61K 31/549; A61K 31/63; A61K 31/519; A61K 31/365; A61K 31/415; A61P 9/12; A61P 29/00; A61K 31/192; A61P 7/10

(52) U.S. Cl. 514/223.5; 514/471; 514/249; 514/473; 514/406; 514/571

(57) ABSTRACT

An oral dosage form of a non-steroidal anti-inflammatory drug (NSAID) is rendered safer for extended or chronic use for anti-inflammatory, analgesic or antipyretic therapy by combining it with an antihypertensive drug. The antihypertensive drug acts prophylactically to inhibit or reduce NSAID-induced hypertension and by so doing the adverse cardiovascular side effects of NSAIDs, such as high blood pressure, heart attack, stroke, opththamologic complications, and death, when NSAIDs are used repetitively over an extended period of time for reducing inflammation or in pain management.

### CROSS REFERENCES TO RELATED APPLICATIONS

This application claims priority to U.S. Provisional Application 61/056,789, entitled “Pharmaceutical Formulations Containing a Non-Steroidal Antiinflammatory Drug and Diuretic” filed on May 28, 2009, and to U.S. Provisional Application 61/097,972 entitled “Pharmaceutical Formulations Containing at least One Non-Steroidal Antiinflammatory Drug and at least One Antihypertensive Drug” filed Sep. 18, 2008, and the complete contents of those applications is herein incorporated by reference.

### BACKGROUND OF THE INVENTION

1. Field of the Invention

This invention is related to the safe provisioning of anti-inflammatory, analgesic and/or antipyretic therapy to human and animal subjects. The invention is also generally related to formulations of non-steroidal anti-inflammatory drugs (NSAIDs) and their use in the prevention or prophylaxis of serious adverse events associated with NSAIDs.

2. Description of the Prior Art

The cyclooxygenase system consists of a ubiquitous set of enzymes that are present throughout the human body and that initiate the biochemical conversion of arachidonic acid into various physiologically active metabolites, including various prostaglandins. These metabolites can increase or decrease immune function, increase or decrease the propensity of blood to clot, or vasodilate blood vessels or vasoconstrict the vessels. They also protect the lining of the gastrointestinal tract against ulceration.

One of the prostaglandins (prostaglandin E) also affects renal (kidney) function. It decreases renal tubular absorption of sodium, thereby increasing sodium excretion; inadequate sodium excretion causes fluid retention, and thereby causes hypertension (high blood pressure). Thus, anything that decreases prostaglandin E production can be expected to increase blood pressure.

Whenever the body suffers an injury, the cyclooxygenase system increases the production of inflammatory metabolites, including prostaglandin E. The increased levels of prostaglandin E then stimulate immune function and inflammation. As a result of these effects, prostaglandin E causes and increases the amount of pain a patient experiences following externally induced injuries such as broken bones, or internally induced injuries such as headaches. It also causes pain in chronic conditions such as arthritis.

In light of the importance of the prostaglandins in the generation of pain, a number of analgesic drugs have been developed over the years to block the production of prostaglandin E, and thereby to decrease the amount of pain the patient experiences. These analgesic drugs, which work by primarily blocking the cyclooxygenase enzyme systems, are referred to as nonsteroidal anti-inflammatory drugs (NSAIDs).

NSAIDs are the most widely consumed pharmaceuticals in the U.S. Although most patients take them for only brief durations of time while treating conditions such as headache or muscle pains, other patients with chronic diseases, such as arthritis, remain on them for months or years.

The NSAID family of pharmaceuticals is divided into two main categories, non-selective NSAIDs and COX-2 inhibitors. NSAIDs that block both the COX-1 cyclooxygenase enzymes and the COX-2 cyclooxygenase enzymes are generally referred to as non-selective NSAIDs. NSAIDs that block preferentially the COX-2 cyclooxygenase enzymes are a specialized category of NSAIDs that are referred to generally as COX-2 inhibitors.

The Merck Manual, 16th Edition, Merck Research Laboratories (1990) pp 1308-1309 provides well known examples of non-selective NSAIDs. These NSAIDs include, but are not limited to, the group consisting of salicylates, indomethacin, flurbiprofen, diclofenac, ketorolac, naproxen, piroxicam, tebufelone, ibuprofen, etodolac, nabumetone, tenidap, alcofenac, antipyrine, aminopyrine, dipyrone, aminopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, ketoprofen, meclofenamic acid, mefenamic acid, niflumic acid, phenacetin, salidifamides, sulindac, suprofen and tolmetin. The salicylates may include acetylsalicylic acid, sodium acetylsalicylic acid, calcium acetylsalicylic acid, salicylic acid, and sodium salicylate.

The non-selective NSAIDs approved by the Food and Drug Administration (FDA) for marketing include indomethacin, ibuprofen, naproxen, etodolac, nabumetone, diclofenac, ketoprofen, piroxicam.

The COX-2 inhibitors include paracoxib, etoricoxib, and lumiracoxib, rofecoxib, valdecoxib, and celecoxib. The COX-2 inhibitors approved by the FDA for marketing include rofecoxib, valdecoxib, and celecoxib.

All of these NSAIDs have been documented to be effective in reducing pain. However, as a result of their inhibition of the synthesis of prostaglandins, NSAIDs can cause multiple serious problems (adverse reactions). A widely recognized adverse reaction resulting from NSAID medications that block the cyclooxygenase enzyme system is gastro-intestinal ulcerations. The FDA has estimated that between 10,000 and 20,000 patients in the U.S. per year die from complications resulting from gastro-intestinal ulcerations resulting from these drugs.

The second category of the NSAID family of drugs, the COX-2 inhibitors, was specifically designed and developed in the hopes that they would be less likely to result in gastro-intestinal ulcerations. The COX-2 inhibitors were specially designed and developed to block only one of the various cyclooxygenase enzymes: The COX-2 enzyme. The COX-2 inhibitors do not block the COX-1 cyclooxygenase enzymes. Since the COX-1 enzymes are present in the lining of the gastrointestinal tract, but the COX-2 enzymes are not, it was anticipated that a drug that selectively blocks the COX-2 enzyme and not the COX-1 enzyme would result in less gastro-intestinal toxicity while still alleviating pain. This was found to be the case and thus, beginning in the late 1990s, FDA approved a number of selective COX-2 inhibitors for marketing in the U.S. including rofecoxib, valdecoxib, and celecoxib.

Recently, the potential for all NSAIDs, including COX-2 inhibitors, to be associated with severe cardiovascular adverse reactions has been recognized. This recognition has led to the drug rofecoxib being removed from the market and to the FDA requiring a boxed warning for all NSAIDs, including COX-2 inhibitors. Boxed warnings are FDA's strongest warnings contained within a drug's labeling. The boxed warning FDA requires for all NSAIDs warns physicians that these drugs may cause myocardial infarctions (heart attacks), and other cardiovascular complications. Currently, the FDA has taken the position that it does not know the etiology by which NSAIDs, including COX-2 inhibitors, increase the risk for myocardial infarctions and other cardiovascular complications. The FDA has noted that the increased rate of myocardial infarctions among patients taking NSAIDs, including COX-2 inhibitors, does not occur until the patients have been receiving the drugs for many months. The FDA has also stated that it is unable to discern any difference in risk among the various approved NSAIDs, including COX-2 inhibitors.

A meta-analysis of randomized clinical trials of various NSAIDs has documented that these drugs increase blood pressure. The amount of the increase reported in this meta-analysis varied slightly depending upon the specific NSAID (no COX-2 inhibitors were reviewed in this meta-analysis). Overall, the increase in mean blood pressure was 5.0 mm Hg. This level of hypertension is less than that seen in essential hypertension but still represents a potential health risk (essential hypertension is the common form of hypertension that occurs for no known reason). For example, an increase in mean blood pressure of only 5.0 mm Hg may not seem excessive, but it has been reported that such an increase, even when occurring for a less than chronic period of time, increased the risk of stroke by 67% and of cardiac disease by 15%. To that end, prevention of NSAID-induced hypertension can be expected potentially to have major benefits to the health of the millions of Americans who take NSAIDs.

This increased risk of hypertension among patients taking various NSAIDs includes the COX-2 inhibitors. Specifically, the FDA approved drug labeling for the COX-2 inhibitor rofecoxib listed high blood pressure as occurring more frequently in rofecoxib treated patients than in placebo control patients. It should be noted that the FDA approved drug labeling for celecoxib also describes an increased rate of hypertension in patients receiving the drug.

The hypertension resulting from NSAIDs can be expected to affect other parts of the cardiorenal system. These other effects would include edema formation and potentially congestive heart failure. It is thus not surprising that a recent epidemiology study conducted using the United Kingdom's General Practice Research Database documented that patients started on NSAIDs had a 1.6 relative risk (a 60% increased risk compared to patients not receiving the drugs) of being diagnosed with clinical heart failure compared to patients of the same age not started on such drugs.

To summarize, clinical data have indicated that the use of any category of NSAIDs, including COX-2 inhibitors, are associated with hypertension, myocardial infarctions, stroke, and death (serious adverse events). However, to date, no effective mechanism for the prevention or prophylaxis of these NSAID-induced serious adverse events has been developed. Furthermore, no effective mechanism for the prevention or prophylaxis of these adverse events has been developed that specifically targets the earliest step in the pathophysiology or etiology of these adverse events, namely the prevention or prophylaxis of NSAID-induced hypertension.

U.S. Patent Publication 2006/0011345 to Spector describes pharmaceutical compositions which includes COX-2 inhibitors and diuretics. The diuretics are identified as being taken in amounts sufficient to maintain the blood pressure of the patient within 5 mm Hg of their average blood pressure. Spector recognizes the advantage of diuretics in maintaining the blood pressure of patients treated specifically and only with COX 2 inhibitors, but does not provide any indication of utility of taking diuretics with non-selective NSAIDs. In addition, Spector provides an indication of utility with specifically and only diuretics, which work by increasing the kidney's excretion of sodium (natriuretics). This results in reduction of fluid in the body which may tend to decrease blood pressure. However, in some instances increasing fluid excretion may not be suitable for proper patient care, which is why there are several other pharmacologic categories of antihypertensives besides diuretics, such as alpha blockers, beta blockers, calcium channel blockers, angiotensin converting factor enzyme inhibitors (ACE inhibitors), and angiotensin-receptor antagonists. Spector does not provide any indication of utility with these other pharmacologic classes of antihypertensive drugs.

U.S. Pat. No. 4,888,343 to Jones and U.S. Pat. No. 5,037,823 to Jones describe pharmaceutical formulations for the relief of dysmenorrhea which include an NSAID in combination both a diuretic and an antihistamine. Similar to Spector, Jones promotes the diuretic as increasing urinary excretion to help alleviate the fluid retention associated with the normal menstrual cycle, and its resulting symptoms, but Jones does not provide any indication of utility of using the diuretic with respect to reducing any type of NSAID-induced adverse reactions.

### SUMMARY

It is an object of the invention to provide safer anti-inflammatory, analgesic or antipyretic therapy to a human or animal subject by providing the subject either concomitantly or in separate dosage forms one or more NSAIDs and one or more antihypertensive drugs.

According to an embodiment of the invention, a pill or capsule includes a therapeutically effective amount of one or more NSAIDs (which can include non-selective NSAIDs and/or COX-2 inhibitors), and one or more antihypertensive drugs in an amount effective to inhibit or prevent cardiovascular side effects normally associated with the NSAIDs that include, but are not limited to, high blood pressure, heart attack, stroke, ophthalmologic complications, and death. The pill or capsule is preferably an oral solid dosage form; however, liquid formulations may also be used. A subject, which can include humans or animals, will orally ingest a pill or capsule multiple times over a course of treatment designed to provide anti-inflammatory, analgesic and/or antipyretic therapy. The pill or capsule contains a therapeutically effective amount of an NSAID to provide the anti-inflammatory, analgesic or antipyretic therapy, but also contains an amount of an antihypertensive (which can include diuretics or non-diuretics) effective substantially to inhibit NSAID-induced cardiovascular side effects such that the NSAID treatment is rendered safer for use. While a single pill or capsule is preferable, the advantages of the anti-inflammatory, analgesic or antipyretic therapy could be achieved using two or more pills containing the NSAIDs and antihypertensives. The methodology may also be employed prophylactically for treating a human patient who is on a therapy known to have significant cardiovascular side effects or is about to begin such a therapy, via concurrent administration of an NSAID (including a COX-2 inhibitor) and one or more antihypertensive drugs in a combination (single) oral dosage form.

### DETAILED DESCRIPTION

Hypertension (high blood pressure) can lead to a number of serious clinical conditions. These include, stroke, myocardial infarction, renal failure, and ophthalmological complications and death. To prevent such serious complications, antihypertensive drugs are widely prescribed. There are multiple types of antihypertensive drugs available to physicians. These include, but are not limited to alpha blockers, beta blockers, calcium channel blockers, angiotensin converting factor enzyme inhibitors (ACE inhibitors), angiotensin-receptor antagonists, and diuretics. Except for the diuretics, these agents work primarily by dilating (relaxing) the body's arteries. As the arteries relax, the tension within the artery decreases and with it the patient's blood pressure. The ability of antihypertensive drugs to reduce blood pressure in patients with hypertension leads to a prevention of myocardial infarction and stroke in these patients. Diuretic drugs, such as thiazides (hydrochlorothiazide and chlorothiazide), loop diuretics (e.g., ethacryinic acid and furosemide), and potassium sparing diuretics (e.g., triamterene), are used to reduce the body's sodium content and thus, concomitantly, its fluid content. As the volume of fluid in the body decreases, blood pressure generally decreases in concert. The invention proposes to use NSAIDs in combination with antihypertensive drugs to provide for a safer anti-inflammatory, analgesic, or antipyretic therapy.

As noted above, it has been clearly established that the use of various NSAIDs, including COX-2 inhibitors, are associated with an increased incidence of various cardiovascular complications: Hypertension, myocardial infarction, congestive heart failure, edema, fluid retention, and death. The myocardial infarctions and deaths may, and probably are, the result of the propensity of these drugs to cause hypertension. A basic premise of the invention is that if the hypertension does cause all the other problems, then most of these complications should be preventable in most patients by the co-administration of an antihypertensive drug acting prophylatically to reduce NSAID-induced hypertension in patients taking any of the various NSAIDs, including COX-2 inhibitors.

By preventing the hypertension over a period of weeks, months, or years, the combining of an antihypertensive drug with the various NSAIDs, including COX-2 inhibitors, can be expected to reduce NSAID-induced hypertension and thus prevent the various sequelae of hypertension, including myocardial infarction, congestive heart failure, and death. This means that the problems the FDA has identified from the use of NSAIDs, problems such as an increased myocardial infarction rate that have led to rofecoxib being removed from the market and a boxed warning being inserted in the labeling of all other NSAIDs, may be prevented.

Antihypertensive drugs have been widely prescribed for many decades and are thus recognized as being generally safe for patients. They have also been documented to decrease the risk of myocardial infarction and stroke in patients suffering from essential (non-drug induced) hypertension (see Lewington, Lancet 360:1903-1913 (2002). Thus, the risk/benefit ratio from adding one or more antihypertensive drugs to an NSAID, including a COX-2 inhibitor, would be potentially significant.

NSAID analgesics are the most widely consumed group of drugs in the US, and one of the most widely consumed drugs worldwide. Thus, reducing the adverse cardiovascular effects of NSAIDs by combination with one or more antihypertensive drugs could potentially be a major benefit for the patients consuming NSAIDs. This is especially the case of patients consuming both prescription and non-prescription non-selective category of NSAIDs (i.e., as opposed to the category of COX-2 inhibitor NSAIDs). For example, since 2006, only a single COX-2 inhibitor (celecoxib) has been marketed in the United States. In contrast, multiple non-selective NSAIDs are marketed in the United States. In part in light of this discrepancy, it has been documented that approximately two-thirds to three-quarters of patients who are prescribed an NSAID by their physician, are prescribed a non-selective NSAID as opposed to a COX-2 inhibitor. Thus, the cardiovascular problems seen with all prescription NSAIDs are potentially a far greater problem from the more widely prescribed non-selective NSAIDs than with the one remaining marketed selective COX-2 inhibitor NSAID.

Non-selective NSAIDs are also far more widely consumed than COX-2 inhibitors since not only are they prescribed by physicians more frequently, but in addition multiple non-selective NSAIDs are marketed as over-the-counter drugs, which are drugs that can be purchased without obtaining a prescription from a physician. In contrast, there are no COX-2 inhibitors that have been approved for over-the-counter sales. Thus, all over-the-counter NSAIDs consumed in the United States are non-selective NSAIDs; no COX-2 inhibitors are consumed from the over-the-counter market.

A combination therapy of one or more NSAIDs and one or more antihypertensive drugs will provide the benefits of the NSAIDs while reducing NSAID-induced serious adverse reactions and should be in compliance with the FDA marketing requirements as necessary. Selection of a suitable combination will depend on a variety of factors including chemistry, patient, and medical application. Evaluation of suitable combinations will involve a review of the chemistry, manufacturing and controls (CMC) investigations, nonclinical (animal) studies, and clinical (human) studies. In some instances, clinical studies will be required to show safety and efficacy. Such studies are well known to those of skill in the art and are summarized below in Example 1.

In preferred embodiments of the invention, the pharmaceutical compositions containing the antihypertensive and the NSAIDs, including COX-2 inhibitors, set forth herein are administered orally. Such oral dosage forms may contain one or both of the drugs in immediate or sustained release form. The oral dosage forms may be in the form of tablets, capsules, troches, lozenges, aqueous or oily suspensions, dispersible powders or granules, emulsions, multiparticulate formulations, syrups, elixirs, and the like.

The combination of the antihypertensive drug and the NSAIDs, including COX-2 inhibitors, can be employed in admixtures with conventional excipients, i.e., pharmaceutically acceptable organic or inorganic carrier substances suitable for oral, parenteral, nasal, intravenous, subcutaneous, internal, or any other suitable mode of administration, known to the art. Suitable pharmaceutically acceptable carriers include but are not limited to water, salt solutions, alcohols, gum arabic, vegetable oils, benzyl alcohols, polyethylene glycols, gelate, carbohydrates such as lactose, amylose or starch, magnesium stearate, talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., other analgesic agents. For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent. Aqueous suspensions containing the above-identified combination of drugs and that mixture have one or more excipients suitable as suspending agents, for example pharmaceutically acceptable synthetic gums such as hydroxypropyhnethylcellulose or natural gums. Oily suspensions may be formulated by suspending the above-identified combination of drugs in a vegetable oil or mineral oil. The oily suspensions may contain a thickening agent such as beeswax or cetyl alcohol. A syrup, elixir, or the like can be used wherein a sweetened vehicle is employed.

### EXAMPLE 1

In some instances human studies may need to be conducted under Investigational New Drug applications (INDs), which will require FDA approval, to show that for certain combinations of drugs and their application, the administration of an antihypertensive drug with NSAIDs, including COX-2 inhibitors, prevents or reduces the adverse affects of the NSAIDs. The human studies will randomize patients to receive either the NSAID, including COX-2 inhibitors, alone, or the NSAIDs, including COX-2 inhibitors, plus the antihypertensive drug. Patients will be administered these drugs for a sufficient period of time necessary to demonstrate the efficacy of the NSAID/antihypertensive drug compared to the control, as required by the definition of the primary endpoint of the clinical trial and any other FDA regulations. Patients will undergo extensive monitoring during that time. The parameters monitored will include blood pressure, with the change in blood pressure during the treatment period being the primary efficacy endpoint. The results between those receiving NSAIDs, including COX-2 inhibitors, alone versus those receiving these drugs plus an antihypertensive drug will be compared using standard biostatistical tests. It will not be necessary to measure the incidence in heart attack, stroke, or death during the treatment period as the primary efficacy endpoint to demonstrate efficacy sufficiently to achieve FDA approval of the New Drug Application (NDA) for the combination drug product.

It is anticipated that the studies will demonstrate that the patients receiving only the NSAIDs, including COX-2 inhibitors, will exhibit more hypertension compared to those receiving NSAIDs, including COX-2 inhibitors, plus an antihypertensive drug. These differences should become apparent within weeks of initiation of the drug therapy. It is also anticipated that the antihypertensive effects of the combination drug product will be recognized by FDA as being associated with a reduced risk of serious cardiovascular events such as myocardial infarction, stroke, and death among patients receiving NSAID, including COX-2 inhibitors, monotherapy compared to those receiving a non-diuretic antihypertensive drug (or in some cases, a diuretic) with the NSAIDs, including COX-2 inhibitors. FDA's recognition of this reduced risk of serious cardiovascular events may be reflected in FDA approved wording of the product labeling.

The antihypertensive drugs currently on the market, and the dosing regimens specified for these drugs in their FDA approved labeling, were approved for treating conditions such as essential hypertension. These conditions, including essential hypertension, are different conditions than the NSAID-induced hypertension proposed to be prevented herein. The prophylaxis or prevention of NSAID-induced hypertension is a new indication for these antihypertensive drugs. It is possible that a different dose of the antihypertensive drugs will be required in order optimally to prevent NSAID-induced hypertension. To that end, clinical studies will also assist in delineating the optimal dose of the non-diuretic antihypertensive drugs (or in some cases, a diuretic).

It is anticipated that the FDA approved labeling for each combination drug products will list in their indications section, not only the current labeling indication for alleviating pain, but also for preventing hypertension and the sequelae of hypertension.

While the invention has been described in terms of its preferred embodiments, those skilled in the art will recognize that the invention can be practiced with modification within the spirit and scope of the appended claims.

1. A method of treating a subject in need of antiinflammatory, analgesic, or antipyretic therapy, comprising the steps of:
providing said subject with multiple doses of one or more non-steroidal anti-inflammatory drugs (NSAIDs) over a predetermined period of time to provide said subject with antiinflammatory, analgesic or antipyretic therapy; and
providing said subject with multiple doses of one or more non-diuretic antihypertensive drugs over said predetermined period of time, wherein said multiple doses of said one or more antihypertensive drugs are sufficient to inhibit or reduce cardiovascular side effects of said one or more NSAIDs.

2. The method of claim 1 wherein each of said providing steps is performed by oral administration.

3. The method of claim 2 wherein said providing steps are preformed simultaneously using a single capsule containing both said one or more NSAIDs and said one or more antihypertensive drugs.

4. The method of claim 1 wherein said one or more NSAIDs includes a non-selective NSAID.

5. The method of claim 1 wherein said one or more NSAIDs includes an NSAID specific for COX-2 inhibition.

6. An oral dosage composition for use in antiinflammatory, analgesic, or antipyretic therapy, comprising:
one or more non-steroidal anti-inflammatory drugs; and
one or more antihypertensive drugs.

7. The oral dosage composition of claim 6 wherein said one or more antihypertensive drugs includes a non-diuretic.

8. The oral dosage composition of claim 6 wherein said one or more antihypertensive drugs includes a diuretic.

9. The oral dosage composition of claim 6 wherein said one or more NSAIDs includes a non-selective NSAID.

10. The oral dosage composition of claim 6 wherein said one or more NSAIDs includes an NSAID specific for COX-2 inhibition.

11. A method for the prophylaxis or prevention of non-steroidal anti-inflammatory drug (NSAID) induced hypertension in a subject, comprising the step of providing to a subject that receives multiple doses of one or more NSAIDs over a predetermined period of time multiple doses of one or more non-diuretic antihypertensive drugs over said predetermined period of time wherein said multiple doses of said one or more non-diuretic antihypertensive drugs are sufficient to prevent or reduce the occurrence of adverse side effects of NSAIDs.

12. The method of claim 11, wherein said providing step is performed by oral administration

13. The method of claim 11 wherein said one or more NSAIDs are non-selective NSAIDs.

14. The method of claim 11 wherein said one or more NSAIDs include an NSAID specific for COX-s inhibition.
